(19) United States
(12) Patent Application Publication
(10) Pub. No.: US 2004/0053266 A1
(43) Pub. Date: Mar. 18, 2004

(54) Protein with cap-and cellulose- binding activity

(76) Inventor: Alexander Belyaev, San Diego, CA (US)

Correspondence Address: BKF JURGENSEN, 800 SILVERADO STREET, 2ND FLOOR, LA JOLLA, CA, 92037, US

(21) Appl. No.: 10/362,778

(22) PCT Filed: Aug. 27, 2001
(86) PCT No.: PCT/US01/26509

(51) Int. Cl. C12Q 1/68; C07H 21/04; C12N 9/42; C12P 21/02; C12N 5/04

(52) U.S. Cl. 435/6; 435/69.1; 435/209; 435/320.1; 435/419; 536/23.2

(57) ABSTRACT

Described are methods and compositions for generating short (cDNA) sequence tags derived from the extreme 5′ ends of eukaryotic mRNAs (“5′ SSTs”). The 5′ SSTs may be aligned with genomic DNA sequences to elucidate the borders of the genes with their corresponding promoters. Thus, the subject invention provides for identification of genes and promoters in genomic DNA sequence and for isolation of nucleic acid molecules encoding same. Vectors comprising such nucleic acid molecules are also provided as are methods of using such nucleic acid molecules, diagnostically, therapeutically and in industrial processes. Storage medium is provided having promoter and gene sequence information in computer readable form stored thereon. In addition, the inventeion provides novel reagents and methods for conducting mRNA expression analysis. The invention also provides reagents and methods for correlating genetic polymorphisms with phenotypic traits of interest.

This application claims priority to U.S. Provisional Patent Application Serial No. 60/228,932, filed Aug. 28, 2000, and to U.S. Provisional Patent Application Serial No. 60/268,552, filed Feb. 14, 2001 both of which are incorporated herein by reference in their entireties.

### FIELD OF THE INVENTION

The present invention relates to the field of biotechnology. More particularly, the present invention provides methods and compositions for generating short sequence tags (SSTs) that have a variety of uses, including, gene and promoter identification, and in methods of genetic analysis.

### BACKGROUND OF TIE INVENTION

A draft of a single composite human genome sequence was recently completed. This new information promises to revolutionize medicine, in part, by allowing scientists and physicians to study how genetic differences between individuals affects the predisposition to, and the course of, disease. Unfortunately, obtaining the genomic sequence data was only the first of many steps needed to transform this information into better diagnostic and therapeutic products.

Gene promoters are the portion of each gene most responsible for regulating the levels at which each gene is active within a particular tissue. As one would expect, genetic polymorphisms in gene promoters play important role in the predisposition to, and in the development of, complex and widespread diseases, such as, coronary heart disease, hypertension, asthma, and Alzheimer's disease, to name a few. Gene promoters themselves are useful as therapeutic products, and can be incorporated into gene therapy and protein expression vectors.

To date, however, few gene promoters have been identified. What is needed are compositions and methods for identifying promoter sequences in the genome, identifying genetic polymorphisms within promoter sequences that are linked to complex diseases, and improved methods for detecting promoter activity.

### BRIEF SUMMARY OF THE INVENTION

The present invention provides compositions and methods for fast and highly reliable prediction of genes and promoters in genomic DNA sequence. In particular, the present invention provides a method for producing and isolating short sequence tags (SSTs) which uniquely identify the border between a gene and its corresponding promoter. The SSTs of the invention will facilitate construction of a comprehensive map of practically all genes and promoters within a genome. Such knowledge will help to focus research into genetic polymorphisms from an unmanageable and cost-prohibitive genome-wide approach to a highly directed and cost-effective regulatory sequence-specific approach. In addition, this invention facilitates the discovery of promoters which, as is discussed further herein, have therapeutic, diagnostic and industrial applications.

In one aspect, the invention provides an isolated protein having both cap-binding activity and cellulose-binding activity. Such a protein may be used both in a free state or when bound to a solid support matrix comprising cellulose. Nucleic acids encoding such a protein are also provided.

In another embodiment the invention provides a method and process for isolating capped mRNA fragments, preferably fragments consisting of between about 10 to 200 nucleotides derived from the extreme 5′ end of eukaryotic mRNAs. In an aspect of this method, eukaryotic mRNA is first fragmented to an average length of between about 20 to about 400 nucleotides, preferably between about 30 to about 200 nucleotides, and most preferably to an average size of about 50 nucleotides; and second those mRNA fragments which are capped are captured with a cap-binding protein bound to a solid support matrix (or “cap affinity support”). Alternatively, capped mRNA can be captured with the cap affinity support first and subsequently cleaved to an average length of between about 20 to about 400 nucleotides, preferably between about 30 to about 200 nucleotides and most preferably to an average size of about 50 nucleotides.

In a related aspect the invention provides a method and process for producing 5′ SSTs comprising (a) isolating capped mRNA fragments, and (b) generating cDNA copies of said fragments. The 5′ SSTs may be transcriptionally oriented by attaching a nucleic acid of known sequence to the 3′ end of the capped mRNA fragments prior to generating cDNA copies of said fragments. Nucleic acids of known sequence may then optionally be added to one or both the 5′ and/or 3′ ends of the cDNA. Thus, for example in one specific embodiment, a poly A sequence is added to the capped mRNA fragments, which are then reverse transcribed using an oligo dT primer, and a poly C sequence is added to the 3′ end of the newly synthesized cDNA. The resulting SST, in this particular example, is transcriptionally oriented and thus may be used to identify which of the two strands of genomic sequence is the coding strand.

Promoter sequences are located immediately adjacent to, and upstream of, the RNA transcriptional start site. Since 5′ SSTs comprise the first few nucleotides of the transcribed message, they can easily be used to identify the border between promoter sequences and transcribed sequences. Thus, in yet another aspect, this invention provides a method of identifying promoter sequences, comprising (a) obtaining 5′ SSTs, (b) aligning the 5′ SSTs with genomic DNA sequences, and (c) recording the promoter sequences.

In still another aspect, the invention provides a method of identifying nucleotide polymorphisms associated with a phenotypic trait of interest, comprising: (a) obtaining DNA samples from a control group and at least one test group wherein the test group has a common phenotypic trait of interest not shared by members of the control group; (b) obtaining at least 200 nucleotides of DNA sequence located immediately adjacent to, and upstream of, the transcriptional start site of a plurality of genes in each individual in both the control and test group; and (c) identifying in said 200 nucleotides of DNA sequence nucleotide polymorphisms which correlate with the phenotypic trait of interest.

In a related aspect, the invention provides a method of identifying nucleotide polymorphisms associated with a phenotypic trait of interest, comprising: (a) obtaining DNA samples from a control group and at least one test group wherein the test group has a common phenotypic trait of interest not shared by members of the control group; (b) pooling the DNA samples from the control group and pooling the DNA samples from the test group; (c) analyzing at least 200 nucleotides of the DNA sequence located immediately adjacent to, and upstream of, the transcriptional start site of a plurality of genes in both the control and test groups for relative abundance of A, T, G or C at each nucleotide position within each group; and (d) identifying in said 200 nucleotides of DNA sequence nucleotide polymorphisms which correlate with the phenotypic trait of interest.

### BRIEF DESCRIPTION OF TIE DRAWINGS

FIG. 1 shows a representation of a preferred embodiment of the method for cloning 5′ SSTs.

FIG. 2 shows examples of primer sequences for amplification of 5′ SSTs.

FIG. 3 shows a representation of junction sequences in the SST concatemer, which allows deduction of transcriptional orientation of the SST orientation and mRNA corresponding strand.

FIG. 4 shows a representation of a microarray having probes specific to the 5′ ends of mRNA and methods for processing an mRNA sample for expression analysis compared to a conventional microarray technology.

### DETAILED DISCLOSURE OF THE INVENTION

The present invention is based on the discovery of novel reagents and methods useful in a highly efficient process of isolating and cloning nucleic acid fragments which correspond to the extreme 5′ ends of capped-eukaryotic mRNA. These reagents and methods will be extremely useful in rapidly identifying gene promoters in eukaryotic genomes.

Promoters regulate the genetic circuitry that controls all aspects of cell and organismal growth and development. Therefore, promoters are targets for therapeutic agents that modulate cell or tissue growth, development, pathogenesis, regeneration or repair by altering, enhancing or reducing the genetic activity of the nucleic acids they regulate (U.S. Pat. Nos. 6,268,144; 5,306,619; 5,693,463; 5,726,014).

Promoters may themselves be used therapeutically, for example, to alter the expression of one or more gene sequences in the human body. Therefore, the invention provides a method of treating a pathological condition in an individual by genetic modification. The method involves contacting a cell of the individual with an effective amount of a targeting construct that includes a promoter and targeting sequences. The targeting sequences correspond to a sequence of a nucleic acid involved in a pathological condition. The targeting construct is taken up by the cell and the promoter is inserted by homologous recombination into the nucleic acid involved in the pathological condition so as to alter its genetic activity. Methods of inserting, removing and replacing nucleic acid sequences at a predetermined location using homologous recombination are known in the art (and described, for example, in Yanez et al., Gene Therapy 5:149-159 (1998), which is incorporated herein by reference in its entirety).

Furthermore, promoters may be useful in gene therapy methods for driving expression of a therapeutic or prophylactic gene. Gene therapy vectors, such as the adeno-associated virus, may be constructed by those of skill in the art that comprise a therapeutic gene driven by a promoter discovered by the methods of this invention (see, for example, WO 99/61601, incorporated herein by reference in its entirety).

Promoters of the invention will also find use in research and in industrial scale protein manufacture when inserted into expression vectors for the production of proteins. Many expression vectors are known to those of skill in the art as are methods of replacing the existing promoters with promoters discovered by the methods of this invention.

In addition, as will be discussed at great length below, knowledge of the promoter sequences will enable the comparative study of regulatory elements between individuals, and groups of individuals, having phenotypic traits of interest. Thus, this invention makes it possible to rapidly and cost-effectively identify the genetic polymorphisms in promoter sequences that contribute, for example, to disease susceptibility.

As used herein, the term “promoter” refers to a single-stranded or double-stranded nucleic acid that promotes expression of a gene, i.e., production of a protein, when present immediately upstream of the gene and in appropriate physiological conditions.

The term “isolated,” when used in reference to isolated nucleic acid molecules or proteins, is intended to mean that the nucleic acid molecule or protein is present in a form or state different from how they are found in nature. Furthermore, when referring to an isolated promoter, it is intended that such a term not read on the promoter when it is merely in the context of an entire chromosome, YAC, cosmid, genomic DNA library, or other such partially isolated nucleic acid preparations that contain numerous other promoter sequences. Such molecules can also be different than molecules found in nature in that they are, for example, produced or expressed by recombinant means or synthesized by chemical means. Furthermore, such molecules can also be different than molecules found in nature in that they are bound or immobilized, with or without cellular constituents, on a filter or solid support.

### EXAMPLES

The illustrative examples provided herein are not intended to be limiting.

### Example 1

Generation of the Cap-Affinity Support.

In one aspect, the invention includes novel fusion proteins that possess affinity for capped mRNA and for a cellulose solid support. It is essential that the fusion protein possess at least these two functions, however, other functional domains may be added as will be appreciated by those of skill in the art.

Many cap-binding proteins are known to those of skill in the art. The cap-binding domain of the fusion protein of this invention may be derived from the known proteins containing cap binding domainsmany of which are shown in Table 1 for the reader's convenience. Mammalian cap binding proteins are preferred and mouse eIF-4E is the most preferred embodiment and is used in this example. The cap-binding domains of such polypeptides, i.e., the portion of the cap-binding protein responsible for cap-binding activity, are well characterized (and described, for example, in Ueda et al., Febs Letters 280: 207-210 (1991); Marcotrigiano et al., Cell 89:951-961, (1997); Quiocho et al., Current Opinion in Structural Biology 10: 78-76 (2000), which are incorporated herein by reference in their entireties).

Thus, by a “fusion protein comprising a cap-binding domain,” the inventor intends that the cap-binding domain is essentially included and not necessarily the entire cap-binding protein. Of course so long as the minimal primary sequence necessary for cap-binding activity is included in the fusion protein other amino acid residues may be included as well, and the entire cap-binding protein may be used. Furthermore, it is possible to include in the fusion protein multiple cap-binding domains, from the same or different cap-binding proteins.

The second essential domain in the fusion protein is a cellulose-binding domain. A large number of cellulose-binding proteins are known in the art. [See, for example, GenBank www.ncbi.nlm.nih.gov/Genbank] The cellulose-binding domain of CbpA (Goldstein, M. A. et al., 1993, J. Bacteriology 175:5762-8) is the most preferred embodiment and is used in this example. The cellulose-binding domains of such polypeptides, i.e., the portion of the cellulose-binding protein responsible for cellulose-binding activity, are well characterized. See, for example, Carrard, G. et al., 2000, Proc. Natl. Ac ad. Sci. USA., 12, 10342-7; Tormo, J. et al., 1996, EMBO J., 15: 5739-51; Lamed R., 1994, J. Mol. Boil., 244: 236-7; Henrissat, B. 1994, Cellulose, 1: 169-196). Thus, by a “fusion protein comprising a cellulose-binding domain,” the inventor intends that the cellulose-binding domain is essentially included and not necessarily the entire cellulose-binding protein. Of course, so long as the minimal primary amino acid sequence necessary for cellulose-binding activity is included in the fusion protein, other amino acid residues may be included as well, and the entire cellulose-binding protein may be used. Furthermore, it is possible to include in the fusion protein multiple cellulose-binding domains, from the same or different cellulose-binding proteins.

In a preferred embodiment the cap-binding domain is arranged carboxy terminally to the cellulose-binding domain, however, either order is permissible. In addition, the fusion protein of this invention may include any number of amino acid residues connecting the two functional domains.

In the methods of the invention, cap-binding proteins fused to solid support binding domains other than cellulose binding domain may be used. For example, fusions with glutathion S-transferase (GST) tag or thireodoxin tag can be constructed. As will be recognized by those of skill in the art, these binding domains may not bind directly to a solid support, however, they can be used in combination with a second molecule which is attached to the solid support and has affinity for the “solid support binding domain.” For example, when using a GST domain is used, glutathion will be bound to the solid support.

Mouse cap binding protein eIF-4E, was isolated from a mouse heart cDNA library (Clonetech, Palo Alto, Calif.). Essentially the entire mouse eIF-4E cDNA was amplified by PCR using the following oligonucleotide primers 5′-GGCGGATCCGACTGTGGAACCGGAAACC-3′ forward and 5′-GCGAAGCTTGCGTGACGAGTCTCCTGT-3′ reverse primer. The generated PCR fragment was cleaved with BamHI and HindIII restriction endonucleases, and cloned into BglII and HindIII digested pET-34b(+) vector which contains the cellulose-binding domain (CBD) of the CbpA gene of Clostridium cellulovorans (Novagen, Madison, Wis.), according to the manufacturer instructions. The plasmid was used to tranform E. coli and clones were grown overnight in 5 ml of LB supplemented with kanamycin, then diluted 1:10 times with the same medium. The cultures were divided into two aliquots. The cells were grown for 1 h at 37° C. on the shaker at approximately 100 rpm/min. IPTG (Sigma, St. Louis, Mo.) was added to one of the aliquots to 0.1 mM concentration and the incubation was continued for another 3-5 hours. The cells were harvested by centrifugation, disrupted by sonication on ice and the proteins were analyzed in SDS-PAGE.

The CBD-eIF-4E fusion protein was prepared as follows: 25 ml of the overnight recombinant E. coli cell culture expressing CBD-eIF-4E fusion protein was diluted to 250 ml with LB medium containing 30 micrograms per ml of kanamycin. The incubation was continued for 1 h at 37° C., after which 150 microliters of 0.3 M IPTG was added and the incubation was continued for another 2 hours. The cells were harvested by centrifugation, washed with cold PBS and disrupted by sonication on ice in a buffer containing 20 mM Hepes pH 7.6, 100 mM KCl, 0.5 mM EDTA and 10% glycerol. The cell lysates were centrifuged for 20 min. at 19,000 rpm in JA-20 rotor of a J-21 centrifuge (Beckman) and the pellet was washed twice with the same buffer, only without glycerol. The pellet contained inclusion bodies with approximately 50% pure CBD-eIF-4E fusion protein. Solubilization and refolding was performed essentially as recommended by Novagen for CBD fusion proteins. Purification of the CBD-eIF-4E fusion protein was performed via attachment to cellulose particles as recommended for CBD fusion proteins by Novagen (1999 product catalog). The purified CBD-eIF-4E fusion protein attached to cellulose particles will sometimes be referred to herein as “cap affinity resin.” The purified fusion protein may be adsorbed to any cellulose support. This cap affinity support which comprises a fusion protein and a cellulose support also forms one aspect of the invention. Particular examples of cellulose support are listed immediately below: magnetisable cellulose/iron oxide low density particles, SCIGEN LID, England; CBinD 100 and CBinD 200 resins, Novagen, Madison, Wis.; cellulose (Sigmacell) types 101, 50 and 20, Sigma, St. Louis, Mo.

Immobilization of cap-binding proteins can be achieved via other methods such as a protein A-immunoglobulin complex described in U.S. Pat. No. 5,219,989 (issued June, 1993, Sonenberg et al. 530/350). In this case, a bifunctional protein is used, which must consist of eIF-4E fused to S. aureus !? Protein A. The Protein A domain mediates attachment to the solid support by binding to immunoglobulin bound thereto. This method suffers a serious disadvantage, however, since the necessary inclusion of additional proteins, i.e. antibodies, results in increased background due to unspecific binding of RNA to these additional proteins. Contaminating RNAse activities were also noted by Sonenberg et al. with some antibody preparations used in the method. Another disadvantage of the technology taught in U.S. Pat. No. 5,219,989, as compared to the present invention, is that attachment of the protein on such columns is not sufficiently strong, which can result in its leaking and can make operating the resin less convenient and repeated use of the resin problematic. Therefore, in the present invention fusion with a cellulose-binding domain is preferred as it provides leak-proof binding to the affinity resin (cellulose). Moreover, cellulose is clean, neutral and inexpensive. Magnetized cellulose particles may also be used as they are more convenient to operate in suspension.

It is important to ensure that the refolded protein retains both cap-binding and cellulose-binding activities in order to ensure satisfactory performance of the affinity resin. To check these activities, an aliquot of the refolded CBD-eIF-4E fusion protein was dialyzed in Buffer A. This aliquot was then brought to a concentration approximately 200-500 μg/ml and subdivided into two aliquots. One aliquot was incubated with 5:1 volume to volume of protein to cap analog resin (7-Methyl-GTP Sepharose 4B commercially available from Amerscham Pharmacia Biotech, Piscataway, N.J.) and the second aliquot was incubated with 5:1 volume to volume of protein to cellulose particles (commercially available from Novagen, San Diego, Calif.). Other sources of cap analog resin and cellulose particles can be used. What is critical is that the protein binding groups in each are provided in molar excess, at least 2× more, to the binding sites of the protein. Each aliquot was incubated at constant rotation for 6-7 hours at room temperature, after which the suspensions were centrifuged and the concentrations of protein remaining in the supernatant was determined and compared with the concentration of protein in the aliquots prior to incubation. It is preferable to use batches of protein, in which at least 30-50% depletion of the protein after incubation on both cellulose and cap analog resin was observed, however, batches in which as little as 20% depletion was observed are acceptable.

Thus, cap-binding and cellulose-binding activity may be assayed according to the method described above. According to this invention, a protein having both cap-binding and cellulose-binding activity is one that exhibits at least 20% depletion in each of the assays described immediately above, preferably at least 30% depletion, more preferably at least 40% depletion and most preferably more than 50% depletion.

### Example 2

Methods of Using Cap-Affinity Support to Isolate Capped mRNA Fragments.

In another aspect, the invention includes a method of isolating capped mRNA fragments, preferably about 20 to 150 nucleotides contained at the extreme 5′ end of eukaryotic mRNAs. As will be appreciated by those of skill in the art when reading this document, this process is useful for many purposes, for example, identifying full-length gene sequences in genomic DNA sequence data, identifying promoter sequences, improving expression analyses, and correlating genetic polymorphisms with disease.

Thus, in this aspect of the invention, there is a method and process for producing capped mRNA fragments, comprising: (a) fragmenting eucaryotic mRNA to an average size of between about 20 to 400 nucleotides in length, preferably between about 30 and 200 nucleotides in length, and most preferably about 50 nucleotides in length; and (b) isolating capped mRNA fragments using a cap affinity support such as the cap binding resin described above.

A. mRNA purification and fragmentation. Total cytoplasmic mRNA from various cell types is purified using conventional techniques (Aulfray and Rougeon, 1980. Eur. J. Biochem., 107, 303-314; Sambrook eat al., 1989; and Molecular cloning: A laboratory manual, Cold Spring Harbor Laboratory Press, Cold Spring Harbor, N.Y., both of which are incorporated herein by reference in their entireties). Alternatively, mRNA from different tissues and organisms at different stages of development can be also purchased from commercial sources such as Clontech Laboratories, Palo Alto, Calif. mRNA fragmentation can be achieved by alkaline hydrolysis or RNase digestion (Linn, S. M. et al., Eds. Nucleases. Second edition. Cold Spring Harbor Laboratory Press, 1993, incorporated herein by reference). RNase digestion is preferred, as it allows digestion in the same buffer as will be used for subsequent cap-affinity purification. RNase immobilized on solid support is used in the preferred embodiment as it can be separated from the RNA by centrifugation, thus allowing better control. Immobilized RNase A is available commercially, for example, from Sigma, St. Louis, Mo.

To monitor the kinetics of RNA fragmentation, aliquots are taken at defined time points and the reaction in each is stopped. Based on the results with small aliquots, the rest of the sample is processed according to the conditions, where the best yield of the fragments of desirable size is observed (most preferably fragments having an average size of about 50 nucleotides in length), however, fragments of other sizes may be used as is indicated above.

B. Isolation of capped mRNA fragments using a cap-binding protein immobilized on solid support (cap affinity support). Preferably, purification is performed in suspension, rather than on columns or other forms of solid support, as it allows one to work with small volumes and quantities of mRNA. Physiological conditions are required for the formation of the capped mRNA-cap binding protein complex. The following conditions are preferred: 20 mM Hepes pH 7.6, 100 mM KCl, 0.5 mM EDTA (Buffer A). It is preferable, therefore, to perform mRNA fragmentation in Buffer A. That way, mRNA fragments can be directly applied to the cap-binding protein following termination of the fragmentation reaction. Preferably, cap affinity resin is incubated in the mRNA fragment/Buffer A cocktail. The adsorbed mRNA (capped fragments) are separated from unadsorbed mRNA (cap-less fragments) by low speed centrifugation, preferably 2000 rpm for 2 min in an Eppendorf microfuge. The resin is resuspended in 20-times volume of Buffer A and pelleted again. This is repeated 2-3 times to achieve thorough removal of unadsorbed RNA fragments. Alternatively, magnetic separation can be used if the solid support is magnetized cellulose.

Capped mRNA fragments may be used for subsequent manipulations while still attached to the solid support. Alternatively, the capped fragments may be eluted from the resin using a number of techniques known in the art, for example, cap analogs can be used (New England Biolabs, Beverly, Mass.). Cap analogs, when provided in excess, can compete out capped mRNA from its complex with the cap binding protein. The competition is achieved at the same conditions as binding, for example, in Buffer A. For example, m7GDP (obtainable from Pharmacia Biotech, Piscataway, N.J.), at a concentration of 0.05-1 mM in Buffer A can be used.

Further mRNA purification steps can be introduced if the purity of the capped mRNA fragments is not satisfactory. Covalent attachment to free amine on a solid support or to molecules allowing further affinity purification, for example, biotin hydrazide can be implemented (see, for example, Seki, M. et al., 1998. The Plant Journal, 15, 707-720, incorporated herein by reference). An additional purification step on boronate resin can be introduced (Wilk H. E. et al. 1982. Nucl. Acids Res., 10, 7621-7633, incorporated herein by reference). This step can be performed before or after the cap affinity purification.

### Example 3

Methods of Identifying Genes and Promoters Using 5′ mRNA Fragments.

In this method, 5′ short sequence tags (5′ SSTs) are generated using the 5′ capped mRNA fragments generated in Example 2. These 5′ SSTs are located at the transcriptional start sites of genes. Thus, by sequencing a 5′ SST and locating an identical sequence in the genomic sequence data, one can identify the junction with the promoter and the transcriptional start site of a gene. Currently, there are only labor-intensive methods for obtaining this information. Most commonly, a gene of interest is identified by analysis of an expressed sequence tag (EST). The EST sequence is extended through labor-intensive procedures, until one obtains a complete cDNA copy of the gene. The cDNA sequence can be aligned with genomic sequence to identify the transcriptional start site and promoter. While EST information is incredibly useful, it is not easy to use this information in a vacuum to determine where gene boundaries are or the location of promoters. In fact, the Eukaryotic Promoter Database, which is the most comprehensive promoter database available in the world, contains the sequence of only a few hundred human promoters. A library of 5′ short sequence tags (“5′ SSTs”) which mark the transcriptional start site of a large number of genes could be combined with EST data and genomic data to vastly improve the mapping of genes and promoters in any eukaryotic genome.

5′ SSTs can range in length from about 8 to about 400 nucleotides. The precise length of the 5′ SST is not critical, however, it should be long enough such that its nucleotide sequence is predicted to be unique in the genome being studied, therefore, for larger genomes one would preferably use longer SSTs. Conversely, it is beneficial to have relatively short SSTs in order to minimize non-canonical nucleic acid-nucleic acid interactions, which may impede performance of the methods of this invention. When studying the human genome, for example, SSTs of between about 20 to 100 nucleotides are preferred.

It is preferable to mark the direction of transcription regarding the 5′ SST sequence in order to elucidate the upstream promoter and the downstream gene elements such as the protein coding sequence.

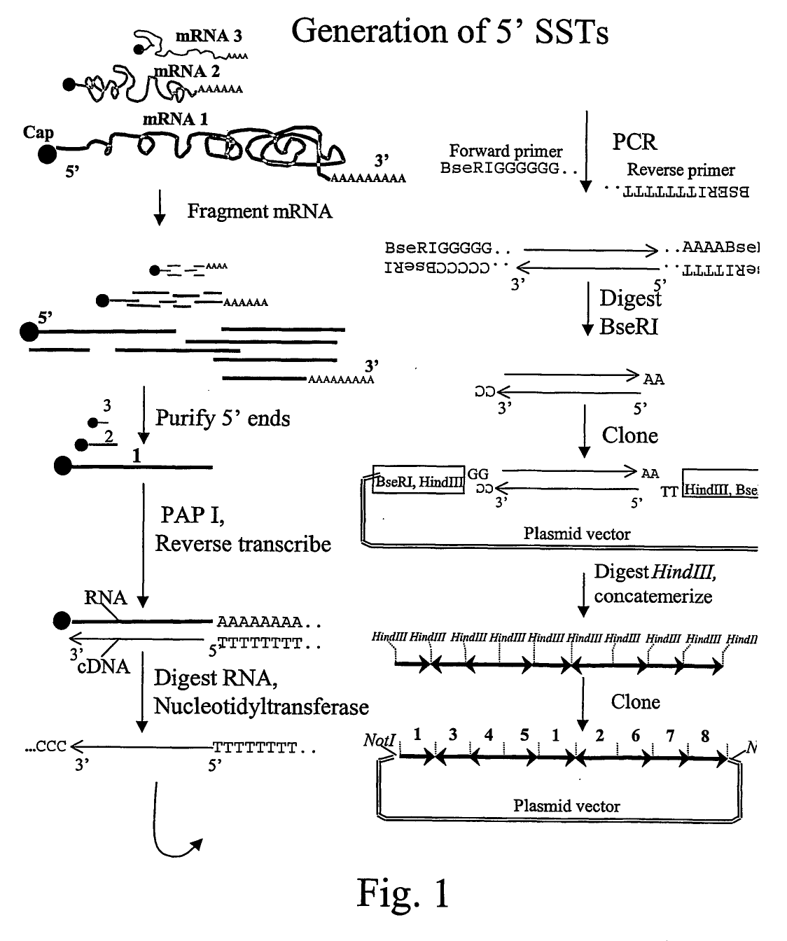

Thus, in one aspect the invention provides a process for producing 5′ SSTs, comprising obtaining capped mRNA fragments; and generating cDNA copies of said fragments. Capped mRNA fragments may be obtained by the methods described above. cDNA copies of said fragments can be obtained by methods known to those of skill in the art, and as briefly described here (FIG. 1).

A. Poly(A) tailing. Preferably, a poly(A) tail is added to the 5′ mRNA fragments. First, it is necessary to remove the 3′ phosphate groups generated by alkaline or RNAse digestion of mRNA, and in order to provide 3′ OH groups needed for chain extension. This is essential, as the enzymatic activity, which adds poly(A) tails to the 3′ ends of the RNA fragments (PAP 1 or poly(A) polymerase), requires 3′ OH group for the substrate recognition. The 3′ terminal phosphate can be converted into 3′ OH group by a phosphomonoesterase, for example, shrimp alkaline phosphatase (Boehringer Mannheim, Indianapolis, Ind.).

5′ mRNA fragments can remain attached to the solid support and poly-A tailed using E. coli poly(A) polymerase (available from Gibco Life Technologies, Rockville, Md.). Poly(A) tailing is preferred, as the reaction is more efficient than with other ribonucleotides. However, poly(C) and poly(U) tailing can also be used. Of course, free 5′ mRNA fragments, not attached to the solid support, can be also used. Importantly, the tailing reaction is not sequence specific, and random tailing of different mRNA species is expected. Poly-A tailed RNA fragments attached via their caps to the resin are separated from the polyA-tailing reaction mixture by centrifugation, or in the case of magnetic beads in a magnetic separator.

B. Reverse transcription. Reverse transcriptases are available from several manufactures. An oligo dT primer, for example, 5′-(T)ₙXY-3′ (where n is a number of T residues, preferably not less than 15, X-A,C or G, and Y-A,C,G or T), can be used to prime the reaction. The reverse transcription reaction is performed according to well-known techniques. Preferably the RNA/DNA hybrid is cleaved with RNAse I, which specifically cleaves single stranded RNA, but not RNA/DNA hybrids. Therefore, incompletely copied mRNA will be cleaved from the cap affinity resin. Complete cDNA copies can be purified from the reaction mixture by pelleting the cap affinity resin using magnetic force or centrifugation, as appropriate.

C. Digestion of RNA in RNA/cDNA hybrids. Digestion of the RNA from the RNA/cDNA hybrids is accomplished using RNase H (Boehringer Mannheim, Indianapolis, Ind.).

D. Transcriptionally orienting the cDNA. In the preferred embodiment, the single stranded cDNA is tailed using nucleotidyltransferase (Boehringer Mannheim, Indianapolis, Ind.). Any deoxytriphosphate, A, T, G, or C, can be used for the reaction. However, in order to establish 5′-3′ delineation poly dG or poly dC should be selected. Alternatively, a template switching oligonucleotide, such as cap finder nucleotide (available from Clontech Laboratories, Palo Alto, Calif.) can be attached to the 3′ end of the cDNA (U.S. Pat. No. 5,962,271). Restriction endonuclease sites can be introduced into the non-conservative part of the template switching oligonucleotide in order to facilitate subsequent cloning. This is done with the same purpose, i.e., attaching a known nucleotide sequence to the 3′ end, which facilitates PCR amplification. If Cap finder is to be used, it has to be attached before the RNAse H digestion step. Another alternative for achieving the same goal is ligation of a single stranded or a double stranded oligonucleotide to the 3′ end of the newly synthesized cDNA.

Thus, the invention further provides a process for producing transcriptionally oriented 5′ SSTs, comprising: (a) obtaining capped mRNA fragments; (b) adding a nucleic acid of known sequence to the 3′ ends of the capped mRNA fragments; and (c) generating cDNA copies of said fragments. Alternatively, this invention my also include a step of adding a nucleic acid of known sequence to the 3′ end of the cDNA.

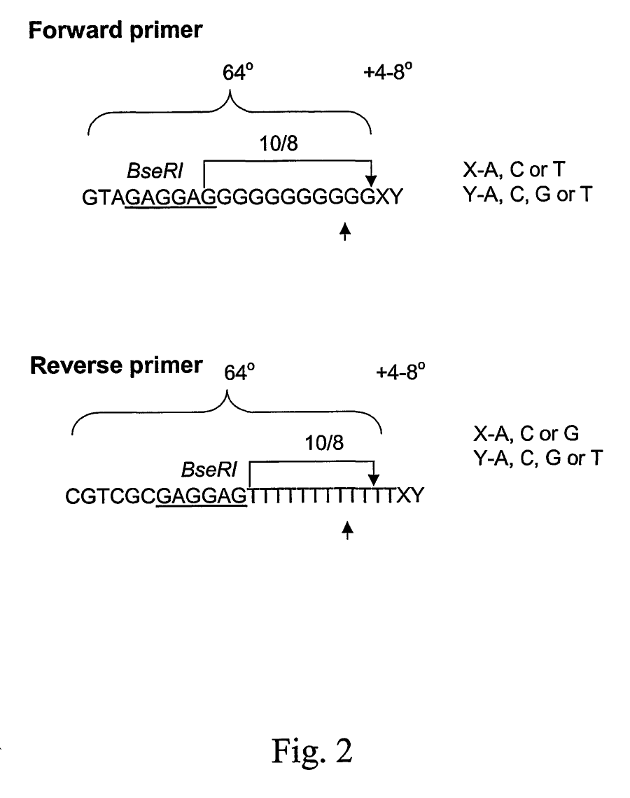

E. PCR amplification. The cDNA copy, which has now been tailed on both ends, may be PCR amplified. The cDNA can be purified from the previous reaction mixture by the ethanol precipitation. However, preferably it is simply diluted into the PCR buffer, as very few cDNA molecules are required for the amplification. Preferably a non-palindromic restriction endonuclease site, which is a substrate for a restriction endonuclease which removes nucleotides adjacent to the restriction site, is introduced at the 5′ end of both primers. It can be the same restriction site in the both primers or a different site in different primers. Examples of such forward and reverse primers are shown on the FIG. 2. The benefit of such a restriction site is that it will remove most of the poly dN tail flanking the 5′ SST. PCR amplification is then performed by known techniques.

F. Digestion of the amplified fragments at their termini. The PCR fragments can be separated from the primers in PAGE, then extracted from the gels and purified. Kits available from Quiagen can be used for this purpose. The purified fragments can be digested with the restriction endonucleases and the digested fragments purified in PAGE as described above. Alternatively, the PCR fragments can be digested first and purified afterwards. These fragments are sometimes referred to as monomeric 5′ SSTs.

Alternatively, SST amplification can also be achieved using T7 polymerase. In this case RNA is amplified using a modification of the procedure described by Van Gelder et al., 1990 and Eberwine et al, 1992, incorporated herein by reference. In essence, RNA amplification is achieved using a primer incorporating a T7 promoter site. Phage SP6 polymerase in combination with SP6 promoter can also be used for this purpose.

G. Cloning monomeric 5′ SSTs. Monomeric 5′ SSTs can be cloned into a specifically designed vector, for example a plasmid vector. Such a vector should contain convenient cloning sites, which after cleavage generate ends compatible with the 5′ SST ends (FIG. 1). The vector can be constructed using commercially available vectors with a convenient selection system, for example pBluescript (Stratagene, La Jolla, Calif.) or pUC series vectors (Amersham Pharmacia Biotech, Piscataway, N.J.), providing blue/white selection on the medium with X-gal and IPTG (Sambrook eat al., 1989. Molecular cloning: A laboratory manual, Cold Spring Harbor Laboratory Press, Cold Spring Harbor, N.Y.). Another cleavage site, for example a 6 or more base pair restriction endonuclease II site, can be designed in the vector in proximity to the cloned fragment.

H. 5′ SST concatemerization and cloning. Concatemerization of monomeric 5′ SSTs is a beneficial step as it improves throughput and reduces the cost of sequencing. Preferably, monomeric 5′ SSTs are cloned into a plasmid, the inserts are cut out with a convenient restriction enzyme and then size-selected.

Next, the monomeric 5′ SSTs are concatemerized using T4 DNA ligase. The ligation of the monomer SSTs into a concatemer can be performed in a reaction mixture containing 1000 units/ml DNA-ligase, 50 mM Tris-HCl (pH 7.5), 10 mM MgCl2, 10 mM dititothreitol, 1 mM ATP and 25 microgram/ml of bovine serum albumin and at least 1 micromolar of the DNA fragments. The ligation is performed at 5-15° C. for several hours, usually up to 12-20 hours. As high as possible concentration of the monomer SSTs is preferred, as this facilitates their concatemerization. Otherwise, predominantly circularization of the fragments instead of their concatemerization will occur. It is also important not to allow the reaction to proceed to completion, as products of the complete reaction are more difficult to clone into a plasmid vector. This is due to the accumulation of ligation products that contain compromised ends. To avoid this problem, the kinetics of the reaction are monitored to elucidate the time point where the reaction is preferably not more than 25-50% complete and when the concatemers reach approximately 500 base pairs in length. A concatemer of 500 base pairs can be confidently sequenced in both directions from the primers complimentary to the flanking regions in the plasmid vector. For this purpose, a small ligation reaction is set up and the time points are taken, for example at 15, 30, 60 minutes, 2, 4, 8, and 16 hours. The time point, which gives reaction products of satisfactory length and, preferably, less than 50% completed concatemerization, is repeated on a larger scale and the products of this reaction are cloned into a sequencing plasmid.

Alternatively, an adaptor capable of terminating concatemerization can be used in a titrated fashion to produce concatemers of any desired length.

The concatemers of desired size, preferably approximately 500 b.p. can be separated from smaller and larger concatemers in agarose gel with subsequent purification. The cloning vectors are preferably adapted to contain rare cutting restriction sites flanking the cloned concatemers. This would allow the concatemerized inserts to be excised from the plasmid, further concatemerized and recloned should sufficient concatemerization (length) not be achieved in the initial attempt.

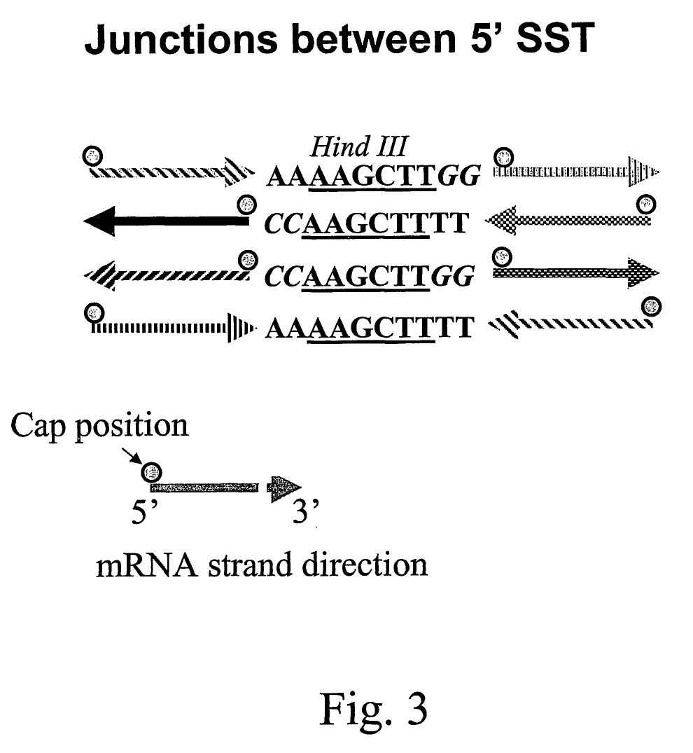

Once the concatemerized 5′ SSTs are cloned they can be sequenced. The individual 5′ SSTs are recognized and recorded, preferably in a fashion indicating the transcriptional orientation. The orientation is determined using known nucleotide sequences at the SST junctions, for example as represented on FIG. 3.

I. Subtractive Hybridization.

The frequency with which any particular 5′ SST appears is expected to correspond with the abundance of the mRNA species in the tissue sample from which it derives. Thus, random sequencing of these SSTs is likely to result in repetitive sequencing of SSTs from highly expressed genes. Rarely expressed genes will be sequenced infrequently or may be missed altogether. To solve this problem, the 5′ SSTs can be used in repetitive rounds of normalization or subtractive hybridization using known techniques. The 5′ SSTs in this case, are the subtractor and can be used to reduce the frequency of 5′ sequences at any step in the foregoing process. In this way, one can penetrate into rarely expressed genes.

5′ SST subtraction works dramatically better for discovering rare mRNA species than known methods due to several synergistically contributing factors. The improvement is partly due to the fact that SSTs are several times shorter than ESTs, and subtractive hybridization methods perform proportionally better on shorter rather than longer nucleic acid sequences, as the non-canonical interactions are drastically reduced with the decrease of the nucleic acid size. Another contributing factor relates to the complexity of the nucleic acid pool. The more complex the nucleic acid pool (total variety of nucleic acid sequences present), the more rare nucleic acids are subtracted along with abundant nucleic acid species. 5′ SSTs are purified from the total mRNA pool, whereas ESTs are not. Therefore, the 5′ SST pool may be as much as 100 times less complex than an EST pool derived from the same organism. Additionally, 5′ SST sequencing throughput can be 10-30 times more efficient than EST sequencing because 10-30 SSTs can be sequenced in a single reaction.

The invention provides a method for isolating 5′ SSTs that correspond to rarely expressed mRNAs, comprising, contacting a first pool of 5′ SSTs with a second pool of 5′ SSTs, incubating the mixture of 5′ SSTs under conditions that promote hybridization, and separating those 5′ SSTs which form double strands from those 5′ SSTs which remain single stranded. There are many modifications that may be made to this method as will be appreciated by those of skill in the art. Rather than 5′ SSTs, for example, one of the two pools of may be comprised of 5′ mRNA fragments. One of the two pools may be bound to a solid support such that nucleic acid hybrids are easily separated from single strands. Alternatively, enzymes that specifically react on double stranded hybrids may be used to eliminate the abundant 5′ SSTs.

The following references provide a number of variations of subtractive hybridization, all of which may be practiced with the 5′ SSTs of the invention. Bonaldo, M. F. et al., (1996) Genome Research, 6: 791-806; Ying, S. -Y and Lin, S. (1999) BioTechniques, 26, 966-979, each of which is incorporated herein by reference in its entirety.

In the preferred embodiment SSTs, which were already sequenced (preferably completely double-sequenced) are used as a subtracter. Synthetic oligonucleotides, derived from the abundant SST sequences can be also used, both approaches can be combined. The SST concatemer has to be fragmented in order to minimize unspecific hybridization. The concatemers can be fragmented into individual monomer SST by appropriate restriction nuclease, for example Hind III, the recognition site for which was designed at the SST junctions (FIG. 1 and FIG. 3). Individual SSTs can be separated from the vector DNA in the polyacrylamide or agarose gels with subsequent purification using an appropriate kit, such as QIAquick gel extraction kit (Quiagen, Inc., Valencia, Calif.) according to the manufacturer instructions. Monomer SSTs also can be excised from the vector and also used for subtraction.

In one specific embodiment, a subtracter can be generated from abundant 5′ SSTs, which can be chemically modified and hybridized to the 5′ SST counterparts in the tester 5′ SST sample. This results in blocking PCR amplification by covalent bonding (Ying, S. -Y. and Lin, S., 1999. BioTechniques, 26, 966-979). Such chemically modified 5′ SSTs can be hybridized to the single stranded cDNA fragments obtained after digestion with endonuclease H and before the PCR amplification step (Example 3E, FIG. 1). Alternatively, they can be hybridized at the next stage, after the first several rounds of the cDNA amplification in PCR. In both cases, the sequences complementary to the modified 5′ SSTs will not be amplified, and thus, they will be effectively depleted from the final SST pool.

The degree to which 5′ SSTs are purified away from non-relevant sequences, such as those derived from the middle of mRNAs, or from other RNA or DNA molecules, is critical for the success of subtractive hybridization. Otherwise, enrichment with non-relevant RNA fragments will occur. The more rare the desired mRNA, the more stringent the purification of 5′ mRNA fragments must be.

### Example 4

Nucleic Acids Comprising Novel Promoter and/or Gene Sequences.

Aligning the 5′ SST sequences generated as described above on linear genomic DNA reveals the 3′ end of a promoter sequence and a 5′ end of the gene it regulates. Thus, in one aspect of the invention, novel nucleic acids are provided each comprising a nucleotide sequence situated starting from the first nucleotide adjacent to, and 5′ of, the transcription start site to about 200 base pairs, preferably about 400, 500, 600, 700, 800, 900, 1000, 1500, 2500, 5000 or 10000 base pairs upstream of the transcriptional start site. In one aspect of the invention such sequences are defined herein as a promoter sequences. The promoter sequences are analyzed for promoter elements, such as TATA boxes and other transcription factor binding sites known to those of skill in the art. This process may be aided through the use of existing promoter algorithms (for example, as described in Prestridge, D. S., 2000, Methods in Molecular Biology, 130, 265-295, incorporated herein by reference in its entirety).

Preferably, promoter sequences are those which posses the ability to promote expression of a gene. One may easily make and test thousands of polynucleotides for promoter activity according to methods well known in the art, such as expressing a reporter gene in physiological environment using a novel promoter. See, for example, Schenborn, E. and Groskreutz, D. (1999), Mol.Biotechnol. 13: 29-44 and Zannis et al. (2001) Front Biosci 6: D456-504, each of which is incorporated herein by reference in its entirety. Minimum requirement for detection of promoter activity is twice the level of the reporter protein over the background activity without promoter with at least 95% confidence.

The 5′ and 3′ non-coding regions of the translated message, which often contain regulatory elements, may also be established and recorded. For example, genomic DNA sequences downstream of the transcriptional start site can be screened for open reading frames, ATG start codons and other well known sequence motifs preceding the ATG start codon in eukaryotes, such as Kozak sequences. (See, for example, Kozak, M.,1996. Mammalian Genome, 7, 563-574, incorporated herein by reference). Similarly, 3′ non-coding regions may be elucidated by screening the genomic DNA sequence upstream of the 3′ end of mRNA, from the 3′ end of mRNA to the stop codon of the open reading frame. Further, both 5′ end and 3′ end sequences are analyzed for intron/exon junctions by methods known to those skilled in the art, and putative introns may be removed from the sequences (Solovyev, V. V. and Salamov, A. A., 1999. Nucl. Acids Res., 27, 248-250).

The complete gene sequences may be deduced using SST sequence information, which is aligned to the linear genomic DNA sequence applying the well-known BLAST algorithm or a similar program. The sequences of internal exons are produced using gene prediction programs, the 5′ ends of the genes are established using 5′ SST sequences, and the 3′ ends using the methods described above and supplemented with EST information from publicly available databases. By this method, and with the essential aid of 5′ SSTs, complete gene sequences may be rapidly generated (see, Solovyev, V. V. and Salamov, A. A., 1999. Nucl. Acids Res., 27, 248-250; Wang, M. S. and Rowley, J. D., 1998. Proc. Natl. Acad. Sci. USA, 95, 11909-11914; Burge, C. and Karlin, S., 1997. J. Mol. Biol., 268, 78-94; Burset, M. and Guigo, R., 1996. Genomics, 34, 353-367, each of which is incorporated herein by reference in its entirety). Methods, combining gene prediction and homology searches, for example GeneWise are preferred (see, Birney E. and Durbin R., 2000. Genome Res 10: 547-8; Guigo et al., 2000. Genome res. 10: 1631-42, both incorporated herein by reference in their entireties).

Thus, the invention also provides nucleic acid molecules comprising novel gene sequences discovered by the method of this invention.

Also provided by the invention are nucleic acid molecules which hybridize, preferably under stringent conditions to nucleic acid molecules consisting of promoter sequences and sequences complimentary thereto, and nucleic acid molecules which hybridize, preferably under stringent conditions to sequences which flank the promoter sequences of this invention, and sequences complimentary thereto. Such sequences are useful for many purposes, for example, as PCR primers for amplifying the novel promoters of the invention.

Stringent conditions are defined as follows: intended overnight incubation at 42° C. in a solution comprising: 50% formamide, 5.times.SSC (750 mM NaCl, 75 mM trisodium citrate), 50 mM sodium phosphate (pH 7.6), 5 times Denhardt's solution, 10% dextran sulfate, and 20 μg/ml denatured, sheared salmon sperm DNA, followed by washing the filters in 0.1 .times.SSC at about 65.degree. C.

### Example 5

Sequence Databases, Sequences in a Tangible Medium, and Algorithms.

The polynucleotide sequences of the promoters and genes of this invention are particularly useful as components in databases useful for search analyses as well as in sequence analysis algorithms. As used in this section entitled “Sequence Databases, Sequences in a Tangible Medium, and Algorithms,” and in claims related to this section, the terms “polynucleotide of the invention” and “polynucleotide sequence of genes and promoters of the invention” mean any detectable chemical or physical characteristic of a polynucleotide of the invention that is or may be reduced to or stored in a tangible medium, preferably a computer readable form. For example, chromatographic scan data or peak data, photographic data or scan data therefrom, called bases, and mass spectrographic data.

The invention provides a computer readable medium having stored thereon promoter and gene sequences of the invention. For example, a computer readable medium is provided comprising and having stored thereon a member selected from the group consisting of: a polynucleotide comprising the sequence of a promoter and/or gene of the invention; a set of polynucleotide sequences wherein at least one of the sequences comprises the sequence of a promoter or gene sequence of the invention; and a data set representing a polynucleotide sequence comprising the sequence of a promoter or gene sequence of the invention. The computer readable medium can be any composition of matter used to store information or data, including, for example, commercially available floppy disks, tapes, silicon chips, hard drives, compact disks, and video discs.

Also provided by the invention are methods for the analysis of character sequences or strings, particularly genetic sequences or encoded genetic sequences. Preferred methods of sequence analysis include, for example, methods of sequence homology analysis, such as identity and similarity analysis, RNA structure analysis, sequence assembly, cladistic analysis, sequence motif analysis, open reading frame determination, nucleic acid base calling, nucleic acid base trimming, and sequencing chromatogram peak analysis.

A computer based method is provided for performing homology identification. This method comprises the steps of providing a first polynucleotide sequence comprising the sequence a promoter or gene of the invention in a computer readable medium; and comparing said first polynucleotide sequence to at least one second polynucleotide sequence to identify homology.

A computer based method is still further provided for polynucleotide assembly, said method comprising the steps of: providing a first polynucleotide sequence comprising the sequence of a promoter or gene of the invention in a computer readable medium; and screening for at least one overlapping region between said first polynucleotide sequence and at least one second polynucleotide or polypeptide sequence.

### Example 6

Novel Microarray Reagents and Methods.

Microarray technology (reviewed in: Watson, A., et al., 1998. Current Opinion in Biotechnology, 9, 609-614), can benefit from the 5′ SST technology taught herein. This includes positional oligonucleotide arrays (Affymetrix, Santa Clara, Calif.), and cDNA printing (Incyte Pharmaceuticals, Palo Alto, Calif.). This also includes arrays produced by other than positional encoding methods, as practiced commercially by Luminex (Austin, Tex.) and Illumina (San Diego, Calif.). This is true in part due to the high quality database of full size genes and regulatory elements, which can be generated by the 5′ SST technology. Even absent the database improvements, however, application of the 5′ SSTs themselves to microarraying procedures will improve the output as will be discussed below. By using 5′ SSTs as probes attached to a chip allows one to use the cap affinity support of this invention to reduce the complexity of the mRNA pool being studied. By removing the majority of the RNA in a sample, one will obtain improved signal to background ratios, improved specificity, sensitivity and reproducibility of the generated data.

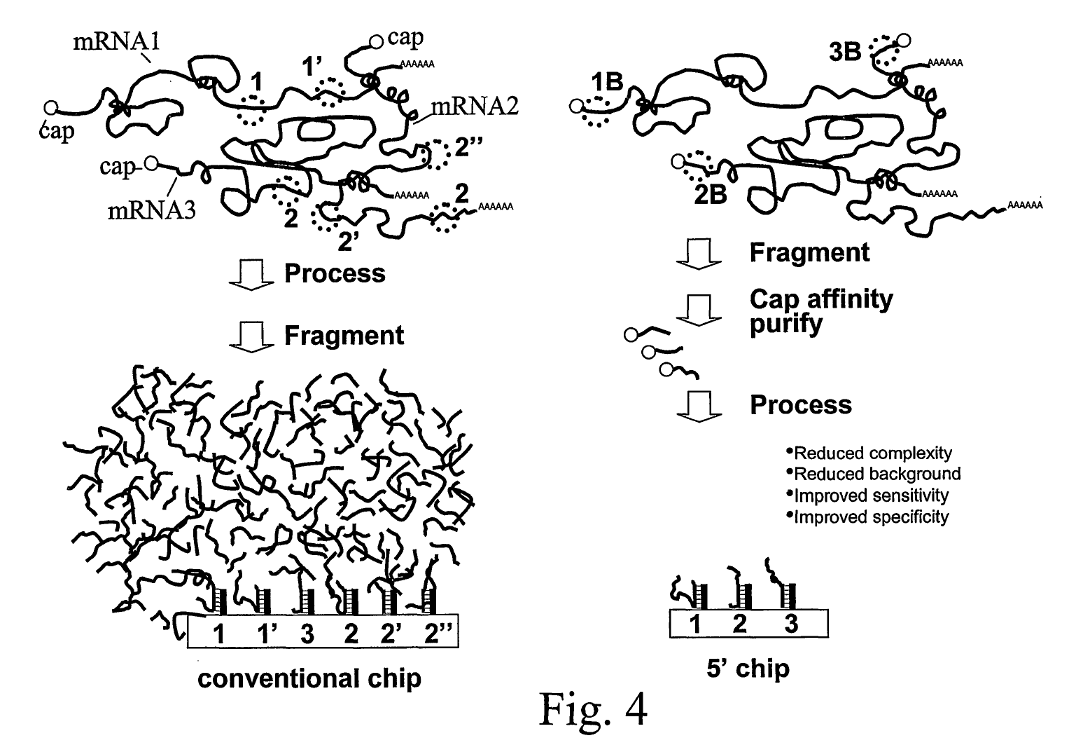

The use of 5′ SSTs and 5′ mRNA fragments in microarray technology is outlined in FIG. 4. The size of the 5′ SST nucleic acid probes immobilized on the microarray support typically can vary from about 10 nucleotides to over 1500 nucleotides. Very short oligonucleotides, those less than about 15 nucleotides in length, are not recommended for analysis of complex mRNA samples, such as samples obtained from human cells since they are likely to cross-hybridize with several mRNA species. Therefore, probes of at least 20 nucleotides are preferred, however, probes which allow reliable “zipping,” for example, probes of 40 to 1000 nucleotides, 40 to 800, 40 to 600, 40 to 400, 40 to 200, 40 to 100 and 40 to 60 are more preferable. It is important that the probes would allow “zipping”, however their size should be kept to the minimum to allow high degree of reduction of complexity of mRNA pool in the analyte due to the cap affinity purification. It is also important that the size of the 5′ mRNA fragments, which are hybridized to the probes, should be of similar size to the probes.

The probes are designed from sequences that are located as close to the 5′ end of the mRNA as possible, however, they should satisfy certain rules for designing probes known to those skilled in the art. Sequence motifs, or similar sequences found in some mRNAs should be avoided for obvious reasons. Likewise, monotonous sequences, consisting largely of one or combinations of two nucleotides should be avoided. For these and other rules, see Lockhart et al., (1996) Nature Biotechnology, 14, 1675-1680, incorporated herein by reference in its entirety. Additionally, in order to increase the signal, probes may be constructed so as to contain repetition of one or more sequences such as branched DNA probes. Alternatively, concatemers of the same 5′ probe can be constructed.

The mRNA sample which is to be analyzed is fragmented, and 5′ ends are purified using the methods described in Example 3. Although boronate resins and other cap-binding reagents may be used to purify the fragmented mRNA, the cap affinity resin described in Example 2 is preferred, since it has better affinity and specificity to the cap structure and will ultimately result in the generation of higher quality microarray data. Cap affinity resin made with magnetic cellulose beads is the most preferred embodiment for use in this process.

The purified 5′ end fragments are labeled with a fluorophore, radioactive isotope or other reporter molecule, which allows detection of the hybridized fragments on the chips (Watson et al., (1998) Current Opinions in Biotechnology, 9, 609-614, incorporated herein by reference in its entirety). The 5′ fragments are contacted with a microarray containing 5′ SST probe under conditions favoring hybridization, microarray is washed and the results read.

Thus, the invention provides a pool of 5′ mRNA fragments labeled with a reporter molecule. The invention also provides a pool of 5′ SSTs labeled with a reporter molecule. In addition, the invention provides a solid support onto which a plurality of 5′ SSTs have been hybridized. The 5′ SSTs may be hybridized randomly, however, the 5′ SSTs are preferably placed on the solid support at a predetermined position. The invention further comprises a method of detecting relative gene expression levels, comprising placing a plurality of 5′ SST probes on a solid support, contacting the solid support with a pool of nucleic acid molecules derived from the 5′ ends of capped mRNA, and quantifying the level of relative abundance of hybridization to each of the 5′ SST probes. Also provided is a kit for assaying relative gene expression, comprising a solid support which has a plurality of 5′ SST probes attached thereto, and a cap affinity support. Such a kit may further comprise means for labeling nucleic acids. In addition, the 5′ SST probes may be individually placed at predetermined locations on the solid support.

### Example 7

Direct Analysis of Gene Expression.

SST technology can be directly applied for quantitative and qualitative analysis of transcripts in various healthy and diseased tissues, in the same way as serial analysis of gene expression (“SAGE”) technology is applied (see U.S. Pat. No. 5,866,330 and Velculescu, V. E. et al., (1995), Science 270: 484-487, both of which are incorporated herein by reference). 5′ SSTs like SAGE tags, originate from distinct places in mRNAs and, therefore, the occurrence of a particular 5′ SST in a concatemer is informative of gene expression.

### Example 8

Detection of Polymorphisms Responsible for Complex Phenotypic Traits.

Genetic polymorphisms account for the phenotypic differences between individuals within a species and between species. Understanding which genotypic differences correlate with phenotypic differences will provide opportunities to improve many aspects of life, such as improved agricultural plants and animals, advanced diagnostic and therapeutic products and improving human health in general. For example, if it were possible to test a healthy person for genetic polymorphisms which are indicative of predisposition to a particular disease, the individual would be able to make life choices (diet, exercise, pharmaceutical intervention, etc . . . ), which would help to postpone or prevent acquisition of the disease. Those of skill in the art will be able to appreciate multiple possible applications from diagnosis and treatment of various human diseases to selection and modification of the attributes of agricultural organisms such as grain yield, protein and starch content, disease and drought resistance.

Existing genetic linkage methods have proven to be highly effective for identifying genetic factors that influence highly penetrant diseases. However, they have proven to be largely inadequate for identifying genetic factors for wide spread complex diseases or traits. Currently, hopes for identification of genes responsible for wide spread diseases and complex traits are largely pinged on genome-wide SNP scans. This approach is similar to traditional genetic linkage mapping with short tandem repeat polymorphism (STRP) markers, with the main difference being that the SNP markers are found much more frequently in the genome, therefore, a more dense coverage with SNP markers can be provided. It remains to be seen if a higher density of SNP markers can significantly improve genetic mapping. Skepticism was raised regarding the feasibility of this approach, since it may not reach the required statistical power to provide meaningful data (Weiss, K. M. and Terwilliger, J. D., 2000, Nature Genetics 26: 151-156). One of the principal weaknesses of the approach is that there is no guarantee that linkage disequilibrium exists in the region of interest and, if there is no disequilibrium, association tests will have no power unless the marker represents an actual functional variant (Borecki I. B. and Suarez, B. K., 2001, Adv. Genet., 42: 4566, incorporated herein by reference in its entirety).

It is advantageous, therefore, to select SNP markers in the regions of the genome that are most likely to contain functional variants. The principle area one would expect to find functional variants is in promoters. Promoter polymorphisms are much more likely to be involved in the development of complex diseases and phenotypic traits than SNPs in other regions of the genome, including protein-coding regions. Here is why. Both simple and complex diseases are caused by alterations in protein expression, which occur at a specific place and time, i.e., in a specific tissue and at a specific period during the lifetime of an organism. Simple diseases often develop very early in life, and are often caused by a complete or nearly complete inactivation of a certain protein, which usually results from drastic mutation of the protein coding sequence, for example, a deletion. Contrary-wise, complex diseases usually develop much later in life. A complex disease often develops following several years of exposure to environmental factors: harsh climate; lack of physical activity; smoking. The development of a complex disease, therefore, is likely to be caused by establishment of a certain gene expression patterns in response to these factors, which in-turn eventually leads to the pathological changes in the organism. These patterns involve complex networks of genes coordinated by the gene regulatory elements. Therefore, it seems that that individual differences in gene regulatory elements, rather than protein mutations can be responsible for individual responses to environmental factors, and predisposition to complex diseases.

The invention provides a method of identifying nucleotide polymorphisms associated with a phenotypic trait of interest, comprising either (a) direct association studies of essentially all polymorphisms in promoter regions of an entire genome or some portion thereof; or (b) using promoter SNPs for genome-wide linkage scans.

A. Direct association studies. Representative groups of test subjects and controls (such as patients and normal volunteers) are selected according to methods known to those skilled in the art (Rao D. C., 2001, Adv. Genet. 42: 13-34; Elston R. C. and Cordell H. J, ibid: 135-150; Gu, C., and Rao, D. C., ibid: 439-458; Cardon, L. R. and Bell, J. I., 2001, Nat. Rev. Genet., 2: 91-99; Zhao, H., 2000, Stat. Methods. Med. Res., 9: 563-87), each of which is incorporated herein by reference in its entirety). In essence, in case-control studies a control group and at least one test group is selected, wherein the test group has a common phenotypic trait of interest not shared by members of the control group. As will be readily apparent to those of skill in the art, these methods can be applied to any eukaryotic organism of interest, and in fact, test and control groups may be selected from separate species. Often, however, it will be beneficial to reduce the number of phenotypic differences (not being studied) between members of the control and test groups so as to reduce the number of genetic polymorphisms that have to be evaluated. Thus, preferably the groups are selected from affected and non-affected family members, which may include parents, grandparents, and children. Alternatively, the groups are preferably selected from non-relatives, which, except for the studied trait or disease, are similar in other respects, for example, gender, age, race and ethnicity.

DNA samples, including pooled DNA samples, can be obtained from the individuals under study according to methods known to those skilled in the art (Wolford J. K. et al., 2000, Hum. Genet., 107: 483487; Shaw, S. H., et al., 1998, Genome Res., 8: 111-23; Giordano, M., et al., 2001, J. Biochem. Biophys. Metods, 47: 101-110; Sasaki, T., et al., 2001, Am. J. Hum. Genet., 68: 214-8; Germer, S. et al., 2000, Genome Res, 10: 258-66; Barcellos L. F. et al., 1997, Am. J. Hum. Genet., 61: 734-47, each of which is incorporated herein by reference in its entirety). In one embodiment, DNA samples obtained from individuals within each group are pooled into subgroups or are combined altogether. Pooled DNA samples can be obtained either by pooling tissue-specific cells, such as lymphocytes, followed by DNA extraction, or by purifying DNA from each individual and then pooling the individual DNA samples. In any case, it is essential that equal representation of each individual in the group be provided in the pooled sample. Pooling samples is advantageous because is allows one to quickly and cost-effectively screen for strongly correlative polymorphisms. Pooling suffers a disadvantage, however, in that weakly correlative polymorphisms may not be detected and haplotypes not discerned.

In one embodiment the promoter sequences under study are amplified by PCR These may be located genome-wide, or restricted to a particular location, such as an individual chromosome or region believed to be responsible for affecting the phenotype of interest. For PCR amplification, one primer is preferably designed to prime amplification within or nearby a 5′ SST sequence of the invention, and a second primer can be designed to primer amplification at least 200 base pairs, preferably at least 300, 400, 500, 1000, 1500 or 5000 base pairs upstream of the 5′ SST. The PCR may include chain terminators useful in a sequencing reaction.

Detection of polymorphisms specific to particular promoter sequences is achieved using methods known to those of skill in the art. These methods include DNA sequencing, kinetic PCR, denaturing high performance liquid chromatography (DHPLC), microarrays, single-strand conformation polymorphism (SSCP) analysis and mass spectrometry-based processes (see, for example, U.S. Pat. No. 6,268,144, Kwok, P. -Y. et al., 1994, Genomics, 23: 138-144; Germer, S. et al., 2000, Genome Res, 10: 258-66; Wolford J. K. et al., 2000, Hum. Genet., 107: 483-487; Kozlowski P. and Krzyzosiak, W. J., 2001, Nucleic Acids Res., 29: E71-1; Sasaki, T., et al., 2001, Am. J. Hum. Genet., 68: 214-8; Fan J. B. et al., 2000, Genome Res, 10: 853-60, each of which is incorporated herein by reference in its entirety).

Furthermore, allele distributions may also be studied according to methods known to those of skilled in the art. Statistical methods for correlating genetic polymorphism markers with a phenotypic trait are known to those of skill in the art (see, for example, Weeks D. E. and Lathrop, M., 1995, Trends Genet., 11: 513-519; Tomlinson, I. P. M. and Brommer W. F., ibid: 493-499; Frankel W. N., ibid: 471-477; Stuber C. W., ibid, 477-481; McCouch S. R. and Doerge, R. W., ibid: 482-487, Elston, R. C. and Cordell, H. G., Adv. Genet., 2001, 42: 135-150; Rice et al., ibid: 99-114, incorporated herein by reference.)

Thus, in this aspect the invention provides a method of identifying nucleotide polymorphisms (genetic polymorphisms) associated with a phenotypic trait of interest, comprising: (a) obtaining DNA samples from a control group and a test group wherein the test group has a common phenotypic trait of interest not shared by members of the control group; (b) obtaining at least 200 nucleotides of DNA sequence located immediately adjacent to, and upstream of, a set of 5′ SSTs corresponding to each individual in both the control and test groups; and (c) identifying nucleotide polymorphisms which correlate in frequency with the phenotypic trait of interest. In a related aspect, the invention provides a method of identifying nucleotide polymorphisms associated with a phenotypic trait of interest, comprising: (a) obtaining pooled DNA samples from a control group and pooled DNA samples from a test group wherein the test group has a common phenotypic trait of interest not shared by members of the control group; (b) analyzing at least 200 nucleotides of the DNA sequence located immediately adjacent to, and upstream of, a set of 5′ SSTs corresponding to both the control and test groups for relative abundance of A, T, G or C at each nucleotide position within each group; and (c) identifying nucleotide polymorphisms which correlate with the phenotypic trait of interest.

B. Genome linkage scans. 5′ SSTs are also useful as markers for genome linkage analyses. Arguably 5′ SSTs are better than randomly selected SST markers currently in use as it is highly beneficial, to locate markers close to genetic polymorphisms that are responsible for disease. The closer the marker, the more tightly linked. If one accepts the proposition that promoter sequences will ultimately be found to be rich with such polymorphisms, locating markers on, or near, promoters has obvious advantages. In this method, the available 5′ SSTs are mapped to the genome being studies. Unusually large gaps left between 5′ SSTs may be filled with prior art SSTs of known location. Linkage analysis studies are performed and analyzed by known techniques.

Throughout this application various publications have been referenced. The disclosures of these publications are hereby incorporated by reference in this application in their entireties in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only be the following claims.

I claim:

1. A protein having both cap-binding activity and cellulose-binding activity.

2. The protein of claim 1, wherein the protein is a fusion protein comprising the amino acid sequence of a cap-binding domain and the amino acid sequence of a cellulose-binding domain.

3. The protein of claim 2, wherein the cap-binding domain is derived from the mouse eIF-4E.

4. The protein of claim 2, wherein the cellulose-binding domain is derived from CbpA.

5. A nucleic acid comprising a nucleotide sequence encoding the protein of claim 1.

6. A vector, comprising the nucleic acid molecule of claim 5.

7. A cap affinity support, comprising
a. the protein of claim 1; and
b. a support matrix.

8. A method of producing capped mRNA fragments, comprising
a. fragmenting eukaryotic mRNA to a size of approximately 8 to 150 nucleotides; and
b. isolating capped mRNA fragments using a cap affinity support.

9. A method of producing 5′ SSTs, comprising
a. obtaining capped mRNA fragments; and
b. generating cDNA copies of said fragments.

10. The method of claim 9, further comprising: adding a nucleic acid of known sequence to the 3′ ends of the capped mRNA fragments prior to generating cDNA copies of said fragments.

11. The method of claim 9 further comprising the step of adding a nucleic acid of known sequence to the cDNA generated in step b.

12. A method of isolating eukaryotic promoter sequences, comprising
a. obtaining transcriptionally oriented 5′ SSTs;
b. aligning the nucleotide sequence of said SSTs with genomic DNA sequence, and
c. synthesizing nucleic acid molecules comprising the sequence adjacent to, and immediately upstream of, the transcriptionally oriented 5′ SSTs.

13. A nucleic acid molecule, comprising the nucleotide sequence of a promoter identified according to the process of claim 12.

14. A vector comprising the nucleic acid molecule of claim 13.

15. A storage medium comprising in computer readable form, the sequence of at least one promoter sequence identified by the method of claim 12.

16. A method of identifying nucleotide polymorphisms associated with a phenotypic trait of interest, comprising
a. obtaining DNA samples from a control group and a test group wherein the test group has a common phenotypic trait of interest not shared by members of the control group;
b. obtaining at least 200 nucleotides of DNA sequence located immediately adjacent to, and upstream of, a set of 5′ SSTs corresponding to each individual in both the control and test groups; and
c. identifying nucleotide polymorphisms which correlate in frequency with the phenotypic trait of interest.

17. A method of identifying nucleotide polymorphisms associated with a phenotypic trait of interest, comprising
a. obtaining pooled DNA samples from a control group and pooled DNA samples from a test group wherein the test group has a common phenotypic trait of interest not shared by members of the control group;
b. analyzing at least 200 nucleotides of the DNA sequence located immediately adjacent to, and upstream of, a set of 5′ SSTs corresponding to both the control and test groups for relative abundance of A, T, G or C at each nucleotide position within each group; and
c. identifying nucleotide polymorphisms which correlate with the phenotypic trait of interest.

18. A method of quantifying the relative abundance of two or more eukaryotic mRNA species in a sample, comprising
a. providing a solid support having at least two nucleic acid probes derived from the 5′ end of a capped mRNA of interest affixed thereto;
b. contacting said solid support with a nucleic acid composition corresponding to the 5′ ends of the mRNA species in the sample under conditions favoring hybridization of nucleic acids having complementary sequences; and
c. quantifying the relative level of hybridization that has occurred to of the nucleic acid probes.

19. A microarray, comprising a solid support having affixed thereto a plurality of nucleic acid fragments substantially identical, or complementary, to the 5′ sequence of a naturally existing eukaryotic mRNA.


SEQUENCE LISTING (5 sequences)

SEQ ID NO: 1 - DNA, 28 bp, Mouse
ggcggatccgactgtggaaccggaaacc

SEQ ID NO: 2 - DNA, 27 bp, Mouse
gcgaagcttgcgtgacgagtctcctgt

SEQ ID NO: 3 - DNA, 21 bp, Artificial Sequence
  Forward primer with BseRI sequence
gtagaggagggggggggnnnn

SEQ ID NO: 4 - DNA, 26 bp, Artificial Sequence
  reverse primer with BseRI sequence
nnnnnnttttttttgaggagcgctgc

SEQ ID NO: 5 - DNA, 10 bp, Artificial Sequence
  Digestion site between concatemers
nnaagcttnn